(19) United States
(12) Patent Application Publication
(10) Pub. No.: US 2022/0215959 A1
(43) Pub. Date: Jul. 7, 2022

(54) SYSTEMS, DEVICES AND METHODS FOR MANAGING USER OUTCOME IN SLEEP THERAPY

(71) Applicants: William Kaigler, Central City, PA (US); Christian Kebekus, Sewickley, PA (US)

(72) Inventors: William Kaigler, Central City, PA (US); Christian Kebekus, Sewickley, PA (US)

(21) Appl. No.: 17/602,447

(22) PCT Filed: Apr. 10, 2020
(86) PCT No.: PCT/US2020/027720

§ 371 Date: Oct. 8, 2021

Related U.S. Application Data
(60) Provisional application No. 62/831,954, filed on Apr. 10, 2019.
(60) Provisional application No. 62/889,131, filed on Aug. 20, 2019.

(51) Int. Cl. G16H 50/20; A61M 16/06; A61M 21/02; A61M 16/00

(52) U.S. Cl. CPC G16H 50/20; A61M 16/0605; A61M 2016/0661; A61M 16/022; A61M 21/02

(57) ABSTRACT

A method of managing sleep therapy includes inputting characterization data associated with each of a plurality of patients into a database system stored in a memory system and inputting outcome data associated with each of the plurality of patients resulting from use of one of a plurality of interface systems in sleep therapy. The method further includes determining a management option for sleep therapy for a person via execution of the algorithm based at least in part upon an optimization of at least one outcome parameter for the person predicted by at least one model of the algorithm based at least in part upon characterization data of the person input into the database system.

### CROSS-REFERENCE TO RELATED APPLICATIONS

This application claims benefit of U.S. Provisional Patent Application Ser. No. 62/831,954, filed Apr. 10, 2019 and U.S. Provisional Patent Application Ser. No. 62/889,131, filed Aug. 20, 2019, the disclosures of which are incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Conventional methods for determining the best medical and other equipment for a user in cases wherein there are a plurality of selections which are affected by plurality of user characterizations/parameters (for example, selecting/fitting a mask for use in positive airway pressure (PAP) treatment) may be very labor intensive and require significant human judgement. Positive airway pressure or PAP treatment is a noninvasive mode of respiratory ventilation used to treat obstructive sleep disordered breathing such as that which occurs in sleep apnea. In the case of interface equipment such as PAP masks or mask systems (for example, mask systems for use in continuous positive airway pressure (CPAP) treatment, bilevel positive airway pressure (BiPAP) treatment, auto-PAP treatment etc.), the inherent complexity of the process, along with a scarcity of feedback regarding the ultimate (clinical) success of the patient has historically resulted in extremely high rates of patient non-compliance (averaging 40% nationally) and high rates of mask return (as high as 35% within the first month).

In current practice, after a patient undergoes a sleep study and, if the patient is diagnosed with sleep apneic events of sufficient frequency and severity to warrant treatment, the patient is typically referred to a trained clinician (typically a respiratory therapists who specializes in sleep apnea) at a local provider of durable medical equipment (DME). The clinician will ask the patient a series of questions which may, for example, include inquiries regarding demographics, inquiries regarding sleep behavior such as favorite sleep position and whether the patient breaths through their mouth during sleep, and health- or wellness-related inquiries such as frequency and severity of allergies, if the patient has ever suffered a broken nose or has a deviated septum, if the patient is claustrophobic, etc. Such questions are typically not recorded by the clinician but are used anecdotally in their assessment regarding the best mask to recommend.

The clinician typically makes an assessment of the optimal size of mask for the patient based upon the anatomical characteristics of the patient's face. This process is manual, sometime using a paper gauge. In many cases, the therapist may choose not to use a gauge at all and will simply make and “educated guess” based upon their judgement of the best size for a particular patient. The patient may have input in this process as well, particularly if the patient has used a PAP mask previously.

The process of measuring anatomical feature of a patient's face has also been automated through the use of image or video data that is analyzed via software. In such methodologies, a three-dimensional image may be required, or a reference object on or near the patient of known dimension(s) may be required in using a two-dimensional image. Moreover, physical features of the patient's face such as the diameter of the iris, which varies little across the human population, have been shown to be suitable for determining physical/anatomical attributes without use of a non-anatomical reference of know dimensions. Measured physical attributes of the patient are compared with stored physical attributes of various masks. Qualifying or disqualifying attributes of the patient (which are typically behavioral) may be used to eliminate options. In general, the software tracks anatomical or other metrics and compares them to known logic or metrics associated with a mask and to rank the masks accordingly.

After a mask system has been selected as described above, the patient is left on their own to take the mask system home and use it. While there are limited monitoring processes which may provide some insight into the ultimate success of the patient, such processes are not well tracked and rarely if ever provide feedback to the clinician who initially selected the mask. In that regard, long term follow up, partly due to the limitations described in the original testing and fitting process and partly due to limitations in previously available technology, have taken two forms. A first process includes manual calls whereby a person calls the patient on a pre-determined schedule to check on their status and, when appropriate, replace their old mask/face interface with a new one for that type of mask to the same type and size. Because the person making the call has very little data, other than what product(s) the patient has previously received and, because there is no knowledge sharing (similar to the therapist interaction described above), there is little or no optimization/improvement in treatment management that occurs with a follow up of this type. The person calling the patient may, for example, be aware of new mask system options but will not have all of the information necessary to recommend a new mask system. In another process, an automated follow up call/contact may be made via phone, SMS or email. In both processes, the interactions with the patient is governed by a pre-determined schedule or a pre-determined call based upon payer requirements and the last-recorded transactions. Patient behavior and/or clinical outcome does not impact the follow up schedule. Such interaction with the patient are rudimentary and are primarily designed to identify significant issues and to replace supplies. Such processes/interactions do not result in recommendations for changes in a mask system or in other treatment parameters. Moreover, such interactions are not typically designed to provide education or feedback to the patient.

Typically, clinicians focus only on new patients, and not existing patients. If a patient has a problem, it is largely up to the patient to alert someone at the DME. In some cases, a patient may not have sufficient information to even recognize a problem. In representative cases in which a problem is recognized by a patient, an alert from the patient via a phone call to the DME may result in the delivery of a substitute mask system (different than the original mask system) for the patient to try. However, there are little or no decision-making criteria available in selecting such a substitute mask system.

### SUMMARY

In one aspect, a method of managing sleep therapy includes inputting characterization data associated with each of a plurality of patients into a database system stored in a memory system and inputting outcome data associated with each of the plurality of patients resulting from use of one of a plurality of interface systems in sleep therapy. Such data may, for example, be received from various sources. The method further includes determining a management option for sleep therapy for a person via execution of the algorithm based at least in part upon an optimization of at least one outcome parameter for the person predicted by at least one model of the algorithm based at least in part upon characterization data of the person input into the database system. The model is developed on the basis of the data associated with each of a plurality of patients and the outcome data associated with each of the plurality of patients. The method may, for example, further include communicating information regarding the management option to the person.

In a number of embodiments, the method includes training at least one machine learning procedure of the model of the algorithm stored in the memory system and executable via a processor system using a training set of the characterization data associated with each of a plurality of patients and the outcome data associated with each of the plurality of patients to create at least one machine learning model and determining a management option for sleep therapy for a person via execution of the algorithm based at least in part upon an optimization of at least one outcome parameter for the person predicted by the at least one machine learning model based at least in part upon characterization data of the person input into the database system. The algorithm may, for example, include a plurality of machine learning procedures. The algorithm may, for example, include a plurality of machine learning models.

The person may, for example, be one of the plurality of patients or be a new patient additional to the plurality of patients. The characterization data of the person and any available output data (for example, resulting from use of one of a plurality of interface systems in sleep therapy), may, for example, be input into the database system and the management option for sleep therapy may be determined via the predicted optimization of the at least one outcome parameter based at least in part upon characterization data and the outcome data of the person input into the database system for the person. The management option may, for example, include determination of a sleep therapy option (for example, a sleep therapy metric such as therapeutic modality, settings for the therapeutic modality such as pressure, selection of an interface system, fitting of a selected interface system etc.) for use by the new patient.

In a number of embodiments, the at least one outcome parameter is a function of outcome metrics including a number of apneas over a predetermined period of time and a usage time over the predetermined period of time. Additional outcome metrics may be included. For example, in a number of embodiments, the outcome metrics further include a level of drowsiness after a defined activity (for example, driving).

The outcome data (or a portion thereof) may, for example, be determined from at least one of data measured from a PAP device, questionnaire data or observed patient behavior. In a number of embodiments, each of the plurality of patients is monitored over time and new data include at least one of new characterization data or new outcome data for each of the plurality of patients is input into the database system over time.

The method may further include updating training of the algorithm based upon the new data for each of the plurality of patients to create at least one updated machine learning model. In a number of embodiments, the method further includes testing each of the at least one updated machine learning model and the at least one machine learning model against a test data set to determine which of the at least one updated machine learning model and the at least one machine learning model has a better confidence interval, and using the one of the at least one updated machine learning model and the at least one machine learning model with the better confidence interval to determine the management option.

In a number of embodiments hereof, the management option includes a change in a sleep therapy option, and the method further includes determining if a recommendation to make the change in the sleep therapy option is to be communicated to the patient based upon a predetermined threshold in a change in the predicted optimization of the at least one output parameter.

In a number of embodiments, the management option includes a recommendation of a therapy option, providing instructions for use of a currently used interface system, a recommendation for an appointment with a physician, a recommendation for a change in lifestyle, a recommendation for a change in sleep behavior, providing education on sleep therapy, or providing positive feedback. The therapy option in a number of embodiments includes one of at least one of a selection of an interface system and fitting of an interface system.

The method may, for example further include, upon occurrence of a triggering event, determining an updated management option for sleep therapy for at least one of the plurality of patients via execution of the algorithm based at least in part upon an optimization of the at least one outcome parameter for the at least one of the plurality of patients predicted by at least one machine learning model determined for use at the time of the triggering event and based at least in part upon the characterization data associated with the at least one of the plurality of patients and the outcome data associated with the at least one of the plurality of patients. The triggering event may, for example, be a passage of a predefined period of time, a request for the at least one of the plurality of patients, receipt of new characterization data associated with the at least one of the plurality of patients or new outcome data associated with the at least one of the plurality of patients, initiation of use of at least one updated machine learning model, use of a new interface system by at least a portion of the plurality of patients.

In a number of embodiment, the method further includes providing a software application on a device of the person which is executable on the device of the person to communicate information between the patient and a remote system including the database system and the algorithm. The device may, for example, be a mobile personal communication device. In a number of embodiments, the method may further include communicating a questionnaire to the person via the software application that is situation sensitive and inputting outcome data determined from a response of the person in the database system. The situation sensitivity may, for example, be determined from data from the device or at least one other device of the person used by the person, and the timing of communicating the questionnaire to the person may be based upon data from the device or the at least one other device. Data from the device of the person or the at least one other device used by the person may, for example, be used to determine that the person is likely to have recently participated in a predetermined activity and the questionnaire includes at least one question inquiring of a level of drowsiness of the person. The situation sensitivity may, for example, be determined from data comprising one or more of motion data, time data and location data (for example, via one or more operationally connected sensors). In a number of embodiments, the predetermined activity is driving.

The characterization data of each of the plurality of patients may, for example, include one or more of anatomical data, sleep behavior data, demographic data, health data, or sleep therapy data. The anatomical data may include at least one anatomical characteristic of the person's head.

In a number of embodiments, the method further includes obtaining a video or an image of the person wearing a currently used interface system. The algorithm may be further configured to determine if the patient is using the interface system incorrectly or non-optimally and to recommend changes or adjustments in use of the interface system or to change the interface system. The algorithm may, for example, include a computer vision procedure to assist in at least one of identifying the interface system or in determining if the patient is using the interface system incorrectly or non-optimally. The method may further include using at least a portion of the interface system as a reference of known dimension.

In a number of embodiment, the method further includes determining at least one anatomical characteristic of the person's head based at least in part on at least one image or video and a known dimensional reference in the at least one image of video selected from an iris of the patient or at least a portion of a sleep therapy interface worn by the patient in the at least one image or video. The at least one image or video may be a two-dimensional image or a two-dimensional video. The two-dimensional image may be supplied by the person electronically. The two-dimensional or other image/video may be analyzed via an image characterization.

The management option in any number of embodiment hereof may, for example, include at least one of selection of an interface system from the plurality of interface systems and fitting of a selected interface system. The management option may, for example, include determining a fitting for headgear of the interface system so that the fitting of the headgear of the interface system is adjusted to the determined fit before delivery.

In another aspect, a system of managing sleep therapy includes a memory system; a processor system in operative connection with the memory system; a database system stored in the memory system, the database system comprising characterization data associated with each of a plurality of patients, and outcome data associated with each of the plurality of patients resulting from use of one of a plurality of interface systems; and an algorithm stored in the memory system and executable by the processor system to determine a management option for sleep therapy for a person based at least in part upon an optimization of at least one outcome parameter for the person predicted by at least one model of the algorithm based at least in part upon characterization data of the person input into the database system. As set forth above, the model is developed on the basis of the data associated with each of a plurality of patients and the outcome data associated with each of the plurality of patients. In a number of embodiments, the system, further includes a communication system (for example, to communicate information to remote persons or system, including, for example, receiving information/data therefrom and communicating information/data thereto such as information regarding the management option).

In a number of embodiments, the algorithm includes at least one machine learning procedure trained using a training set of the characterization data associated with each of a plurality of patients and the outcome data associated with each of the plurality of patients to create at least one machine learning model, wherein the algorithm determines a management option for sleep therapy for a person based at least in part upon an optimization of at least one outcome parameter for the person predicted by the at least one machine learning model based at least in part upon characterization data of the person input into the database system. The system may include a plurality of machine learning procedures. The system may include a plurality of machine learning models.

The person may be one of the plurality of patients or may be a new patient additional to the plurality of patients. The system may, for example, further include a communication system via which information, which may include management options, is communicated between patients and the system.

The characterization data of the person and any available output data (for example, resulting from use of one of a plurality of interface systems in sleep therapy), may, for example, be input into the database system and the management option for sleep therapy may be determined via the predicted optimization of the at least outcome parameter based at least in part upon characterization data and the outcome data of the person input into the database system for the person. The management option may, for example, include determination of a sleep therapy option (for example, a sleep therapy metric such as therapeutic modality, settings for the therapeutic modality such as pressure, selection of an interface system, fitting of a selected interface system etc.) for use by the new patient.

In a number of embodiments, the at least one outcome parameter is a function of outcome metrics including a number of apneas over a predetermined period of time and a usage time over the predetermined period of time. Additional outcome metrics may be included. For example, in a number of embodiments, the outcome metrics further include a level of drowsiness after a defined activity (for example, driving).

The outcome data (or a portion thereof) may, for example, be determined from at least one of data measured from a PAP device, questionnaire data or observed patient behavior. In a number of embodiments, the algorithm is configured to monitor each of the plurality of patients over time and new data includes at least one of new characterization data or new outcome data for each of the plurality of patients is input into the database system over time.

In a number of embodiments, the algorithm is configured or executable to update training of the algorithm based upon the new data for each of the plurality of patients to create at least one updated machine learning model. The algorithm is configured or executable to test each of at least one updated machine learning model and the at least one machine learning model against a test data set to determine which of the at least one updated machine learning model and the at least one machine learning model has a higher confidence interval; and use the one of the at least one updated machine learning model and the at least one machine learning model with the higher confidence interval to determine the management option.

The management option may, for example, include a change in a sleep therapy option, and the method further comprises determining if a recommendation to make the change in a sleep therapy option is to be communicated to the patient based upon a predetermined threshold in a change in the sleep therapy option in the predicted optimization of the at least one output parameter. The management option may, for example, include a recommendation of a therapy option, providing instructions for use of a currently used interface system, a recommendation for an appointment with a physician, a recommendation for a change in lifestyle, a recommendation for a change in sleep behavior, providing education on sleep therapy, or providing positive feedback. The therapy option may, for example, include at least one of selection of an interface system of fitting of a selected interface system.

The algorithm may, for example, be further configured, upon occurrence of a triggering event, to determine an updated management option for sleep therapy for at least one of the plurality of patients via execution of the algorithm based at least in part upon an optimization of the at least one outcome parameter for the at least one of the plurality of patients predicted by at least one machine learning model determined for use at the time of the triggering event and based at least in part upon the characterization data associated with the at least one of the plurality of patients and the outcome data associated with the at least one of the plurality of patients. The triggering event may, for example, include a passage of a predefined period of time, a request forth the at least one of the plurality of patients, receipt of new characterization data associated with the at least one of the plurality of patients or new outcome data associated with the at least one of the plurality of patients, initiation of use of at least one updated machine learning model, use of a new interface system by at least a portion of the plurality of patients. The system may be otherwise characterized as described above and elsewhere herein.

In another aspect, a non-transitory computer readable storage medium having instructions stored thereon, that when executed by a processor, perform actions including: storing input of characterization data associated with each of a plurality of patients into a database system; storing input of outcome data associated with each of the plurality of patients resulting from use of one of a plurality of interface systems in sleep therapy; training at least one machine learning procedure of an algorithm using a training set of the characterization data associated with each of a plurality of patients and the outcome data associated with each of the plurality of patients to create at least one machine learning model, determining a management option for sleep therapy for a person via execution of the algorithm based at least in part upon an optimization of at least one outcome parameter for the person predicted by the at least one machine learning model based at least in part upon characterization data of the person input into the database; and communicating information regarding the management option to the person. The non-transitory computer readable storage medium may be otherwise characterized as described above and elsewhere herein.

In another aspect, a method of collecting data from a person in sleep therapy includes providing a software application on a device of the patient which is executable on the device to communicate information between the person and a remote system comprising a database system, communicating a questionnaire to the patient via the software application that is situation sensitive, and inputting outcome data associated with the sleep therapy determined from a response of the patient in the database system. The device may, for example, be a mobile personal communication device. Situation sensitivity may, for example, be determined from data from the device or at least one other device used by the person and the timing of communicating the questionnaire to the person is based upon data from the device or the at least one other device. Data from the device or the at least one other device may, for example, be used to determine that the person is likely to have recently participated in a predetermined activity, and the questionnaire includes at least one question inquiring of a level of drowsiness of the person. Situation sensitivity may, for example, be determined from data including one or more of motion data, time data and location data. The predetermined activity may, for example, be driving.

In a further aspect, a personal communication device for use by a patient of sleep therapy includes a memory system, a processor system in operative connection with the memory system, a communication system in operative connection with the processor system and the memory system; and an algorithm stored on the memory system and executable by the processor system to communicate information between the patient and a remote system comprising a database system comprising data of the patient's sleep therapy, the algorithm being configured to transmit patient responses to a questionnaire to the patient that is situation sensitive to the remote system, to provide outcome data associated with the sleep therapy determined from a response of the patient to the remote system for storage in the database system. The system may be otherwise characterized as described above and elsewhere herein.

In still a further aspect, a method of determining at least one characteristic of at least one of a patient's head or a sleep therapy interface system includes obtaining at least one image or video of the patient wearing the sleep therapy interface system, and determining the at least one characteristic based at least in part on a known dimensional reference comprising at least a portion of a sleep therapy interface worn by the patient in the at least one image or video.

The present devices, systems, and methods, along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates a representative embodiment of a screen capture in which software hereof is used to determined dimensions of a patient's head (including dimensions of the patient's face) using the patient's iris as a dimensional reference point.

FIG. 2 illustrates a representative embodiment of a system hereof and communication pathways with other devices.

FIG. 3A illustrates another representative embodiment of data communication in a system hereof.

FIG. 3B illustrates characterization data and clinical outcome data for entry into a database hereof and several recommendations for management/treatment options determined by an algorithm hereof for several representative patients.

FIG. 4 illustrates a number of embodiments of (clinical) outcome parameters for use in an algorithm/model hereof which is executable to predictively optimize at least one such outcome parameter, which is referred to as a Sleep Quotient or SQ in the embodiments of FIG. 4.

FIG. 5 illustrates an embodiment of a compliance report from a PAP device for a representative patient from which data of clinical outcome is available.

FIG. 6 illustrates a flow chart of a representative embodiment of a methodology hereof.

FIG. 7A illustrates a flow chart of a representative embodiment of a methodology for data collection hereof.

FIG. 7B illustrates a flow chart of a representative embodiment of a methodology for training one or more machine learning algorithms to develop a model for determining a predicted value or values of one or more metrics resulting from a predicted optimization of the outcome parameter.

FIG. 7C illustrates flow charts of representative embodiments of standard techniques for preprocessing of data and sleep-therapy related techniques for preprocessing of data for use in one or more machine learning algorithms hereof.

FIG. 7D(i) illustrates a portion of a flow chart of a representative embodiment of a methodology for predicting metric(s) (for example, therapeutic metrics) a single person or patient using a model developed via one or more machine learning algorithms hereof which determines values of predicted metrics on the basis of or associated with optimization of an outcome parameter.

FIG. 7D(i) illustrates the remaining portion of the flow chart of FIG. 7D(i).

FIG. 7E illustrates a flow chart of a representative embodiment of logic for a methodology of providing information to a patient including, for example, recommendations for actions or changes.

FIG. 7F illustrates a flow chart for a representative embodiment of image/picture taking assistance algorithm hereof.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a database” includes a plurality of such databases and equivalents thereof known to those skilled in the art, and so forth, and reference to “the database” is a reference to one or more such databases and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise clearly contraindicated by the text.

The terms “electronic circuitry”, “circuitry” or “circuit,” as used herein include, but are not limited to, hardware, firmware, software or combinations of each to perform a function(s) or an action(s). For example, based on a desired feature or need. a circuit may include a software-controlled microprocessor, discrete logic such as an application specific integrated circuit (ASIC), or other programmed logic device. A circuit may also be fully embodied as software. As used herein, “circuit” is considered synonymous with “logic.” The term “logic”, as used herein includes, but is not limited to, hardware, firmware, software or combinations of each to perform a function(s) or an action(s), or to cause a function or action from another component. For example, based on a desired application or need, logic may include a software-controlled microprocessor, discrete logic such as an application specific integrated circuit (ASIC), or other programmed logic device. Logic may also be fully embodied as software.

The term “processor,” as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as random access memory (RAM), read-only memory (ROM), programmable read-only memory (PROM), erasable programmable read only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

The term “controller,” as used herein includes, but is not limited to, any circuit or device that coordinates and controls the operation of one or more input and/or output devices. A controller may, for example, include a device having one or more processors, microprocessors, or central processing units capable of being programmed to perform functions.

The term “logic,” as used herein includes, but is not limited to. hardware, firmware, software or combinations thereof to perform a function(s) or an action(s), or to cause a function or action from another element or component. Based on a certain application or need, logic may, for example, include a software controlled microprocess, discrete logic such as an application specific integrated circuit (ASIC), or other programmed logic device. Logic may also be fully embodied as software. As used herein, the term “logic” is considered synonymous with the term “circuit.”

The term “software,” as used herein includes, but is not limited to, one or more computer readable or executable instructions that cause a computer or other electronic device to perform functions, actions, or behave in a desired manner. The instructions may be embodied in various forms such as routines, algorithms, modules or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software is dependent on, for example, requirements of a desired application, the environment it runs on, or the desires of a designer/programmer or the like.

As used herein, the term “personal communications device” refers to a device (typically portable or mobile device) which includes a communication system, a processor system, a user interface system (for example, a visual feedback system including a touchscreen or other display, an auditory feedback system, and a tactile feedback system, an user input system etc.) and an operating system capable of running general-purpose applications. The personal communication system may also include a camera system for taking images and/or video. Examples of personal communications devices include, but are not limited to, smartphones, tablet computer, computers and custom devices. As used herein, the term “tablet computer” or tablet, refers to a mobile computer with a communication system, a processor system, at least one user interface as described above (typically including a touchscreen display), and an operating system capable of running general-purpose applications in a single unit. As used herein, the term “smartphone” refers to a cellular telephone including a processor system, at least one user interface as described above (typically including a touchscreen display), and an operating system capable of running general-purpose applications. Mobile personal communication devices are typically powered by rechargeable batteries and are housed as a single, mobile unit. Moreover, in a number of embodiments personal communications devices are able accept input directly into a touchscreen (as opposed to requiring a keyboard and/or a mouse). Personal communications devices as typically provide for internet access through cellular networks and/or wireless internet access points connected to routers. A number of representative embodiments of systems and/or methods hereof are discussed in connection with the user of a smartphone as the personal communication device.

In a number of embodiments, sleep therapy management devices, systems and methods hereof facilitate management of the treatment of sleep therapy patients through determining/recommending sleep therapy management options in, for example, the selection of optimal interface equipment (for example, a PAP mask system) for patients, the ongoing monitoring of the patient after interface selection and fitting, the real-time and long-term optimization of the interface selection/fitting and the overall management of the patient's sleep treatment. As used herein, the term “fitting” refers, for example, to determining the size of the interface/mask system as well as determining adjustment of headgear via which the system is retained on the user's head. In a number of embodiments, a database system including one or more databases includes patient characterization data metrics and clinical outcome data metrics for each of a plurality of patients. In the algorithms hereof, one or more clinical outcome or outcome parameters (which may be defined via one or more clinical outcome metrics or functions thereof) are defined to quantify patient clinical outcome/results associated with sleep therapy. In a number of embodiments, one or more machine learning and or other algorithms may, for example, be used in connection with the database system which are trained/designed (using includes patient characterization data and clinical outcome data for a plurality of patients) to provide a model to determine optimized (initially and/or over a long term) values for one or more defined management options for sleep therapy (for example, therapeutic/clinical and/or behavioral metrics) to achieve optimization of one or more outcome parameters for a particular patient. Data or metrics for patients may be collected/determined using monitoring and surveying devices and associated software to provide initial optimization in, for example, selecting and fitting sleep therapy equipment and/or for a long-term optimization loop for each of a plurality of patients.

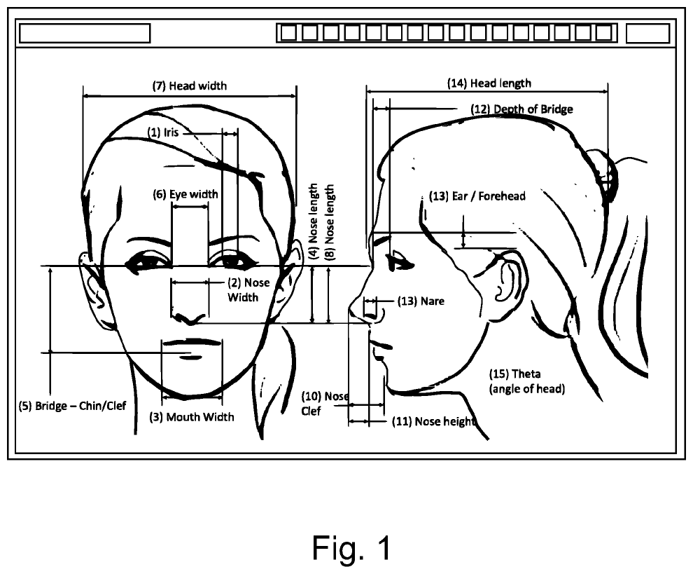

Data characterizing a patient or the patient's sleep therapy (sometimes referred to herein as “patient characterization data”) includes anatomical data of a patient's head/face and other data relative to sleep treatment/therapy. As described further below, one or more photographic or video images of the patient's head/face may be used for collecting/determining anatomical characterization data as illustrated in FIG. 1. Further, non-anatomical characterization data for the patient may also be collected for entry in the database system via answers to a series of questions. A questionnaire or survey may, for example, be administered by a therapist or may be delivered to a patient via a communication system (for example, via an app or internet browser). In a number of embodiments of the present devices, systems and methods, the patient characterization data includes, for example, data on anatomical features/dimensions as described above, data of the patient's sleep behavior, data of the patient's demographics, data of the patient's health or wellness (both physiological and mental health/wellness), and/or data of a patient's current or previous experience with PAP therapy (for example, therapeutic modality, pressure, interface, interface fitting, etc.).

Patient characterization data other than anatomical data may for example, be obtained via questions posed to patients as well as to therapists. Representative questions to patients, may for example, include:
1. Patient age
2. Patient weight
3. Patient sex
4. Do you ever fall asleep while wearing eyeglasses?
5. Do you have dentures?—If, yes, do you wear them when you sleep?
6. What is your favorite position to sleep in (side, back, stomach, sitting up)?
7. Do you notice mouth dryness after sleeping?
8. Do you ever experience dryness in the eyes after a night of using your PAP therapy?
9. Do you feel claustrophobic (when wearing a mask)?
10. Do you have issues with chronic sinus congestion or allergies?
11. Do you use dental apparatus when you sleep (to treat TMJ or teeth grinding)?
12. Do you have a pacemaker?
   Representative questions or requests for information to pose to the therapist may, for example, include:
1. If the patient has used other PAP interfaces/masks before:
   a. Does the patient know which mask they have been using (input manufacturer, model and size)?
   b. Was the patient satisfied with the mask that they used in the study?
   c. If not, why not?
      i. Claustrophobic feeling
      ii. Not getting enough air
      iii. Coldness around the corner of his/her eye
      iv. Noticed leak
      v. Discomfort (forehead, face, other)
   d. Patient requests a new (different) mask
2. Prescribed pressure (continuous, inspiratory, expiratory)
3. Modality (CPAP, Auto or Smart PAP, Bi-PAP, V-PAP, other)
4. Service type (Exchange, Re-fit, Re-PAP)
5. Broken nose or deviated septum
6. Was the mask selected or prescribed by the physician?

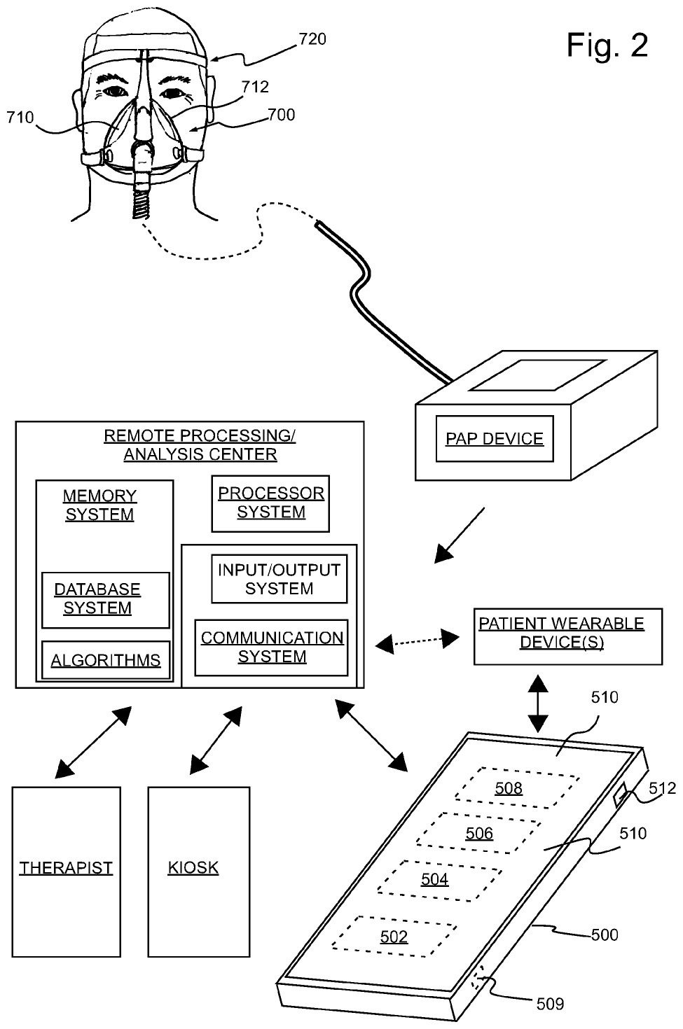

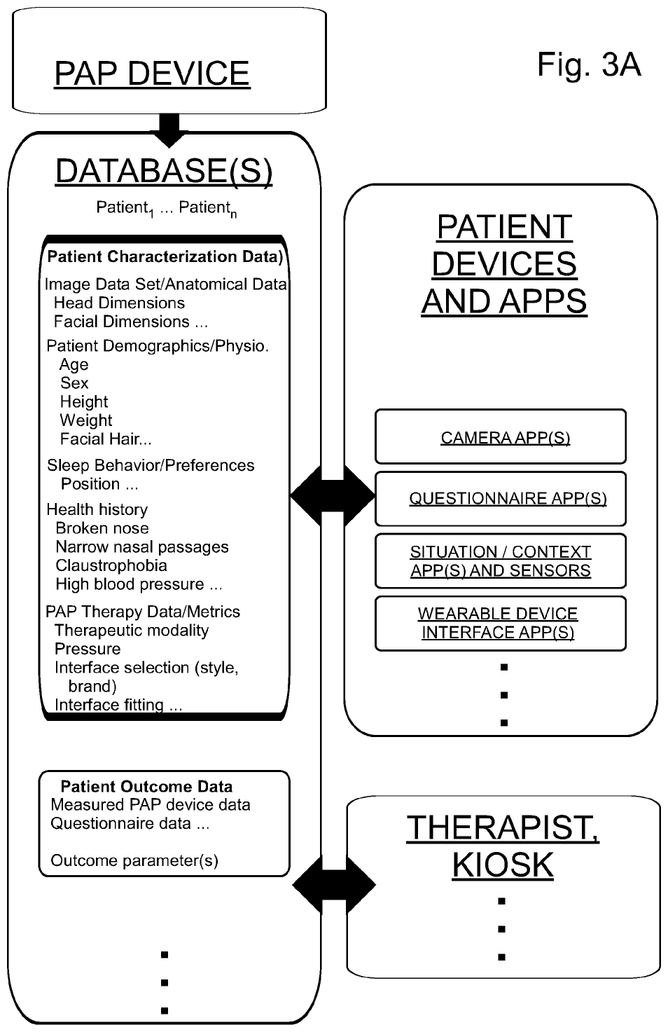

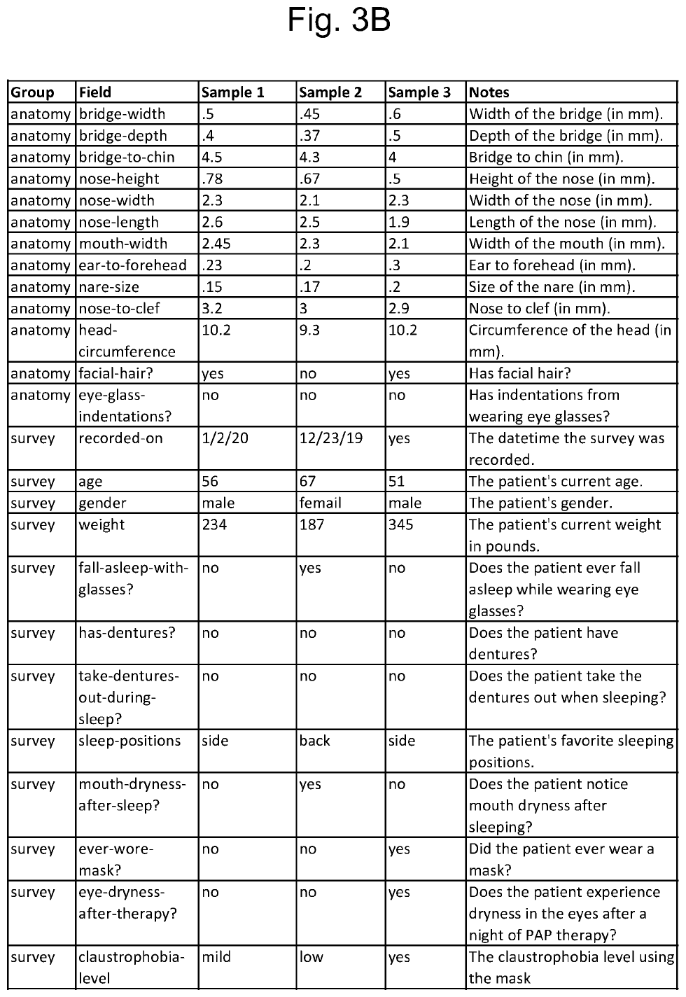

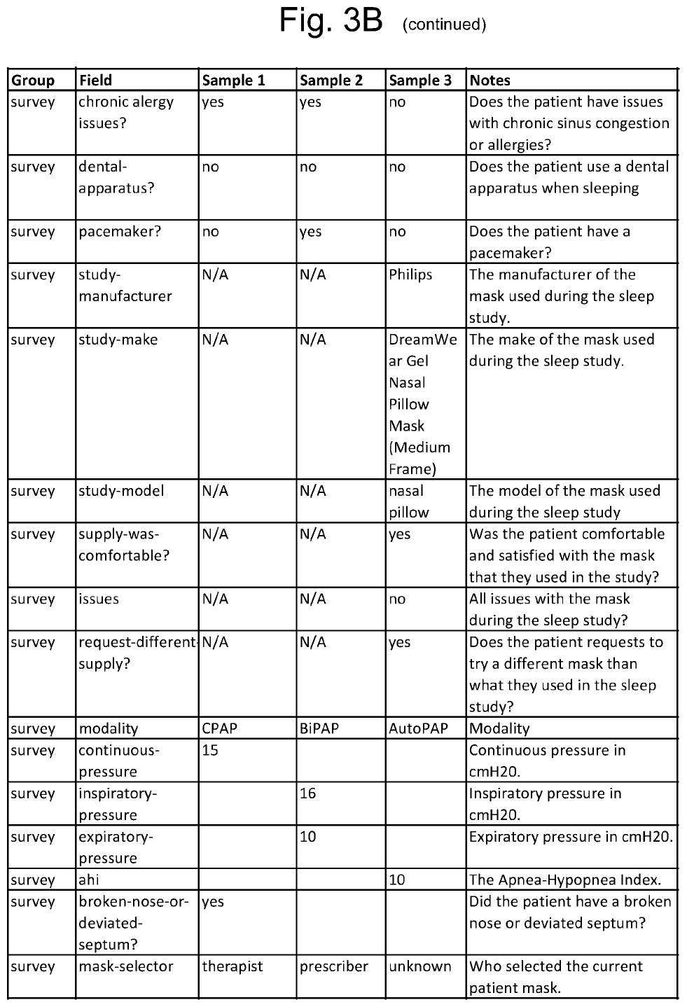

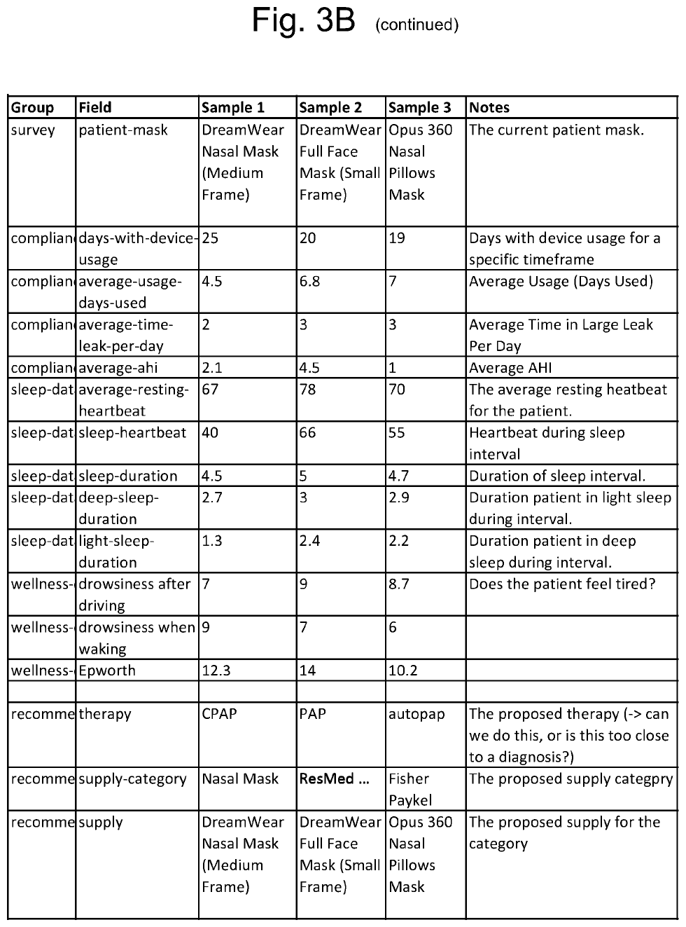

FIGS. 2 and 3A illustrates schematically the communication of data to and from a remote system or processing/analysis center which includes a memory system upon which one or more databases of a database system hereof are stored. An input/output system provides for communication between the remote system and users and/or other devices (for example, using communication protocols as known in the art via landline telephone connections, wireless telephones connectivity, broadband internet connectivity and/or other communication/connectivity channel(s) systems as known in the communication arts, which are represented schematically by arrows in FIG. 2). For example, a wired or wireless communication system may provide for communication between the remote processing center and a number of devices such as a PAP device, a patient wearable device (for example, a FITBIT® device (available from Fitbit, Inc. of San Francisco, Calif., United States), an APPLE® Watch (available from Apple, Inc. of Cupertino, Calif., United States), etc.), a personal communication device (for example, a smartphone), a patient computer, a photograph/information kiosk, a therapist device etc.

Anatomical data of a patient's head/face for user herein may, for example, be obtained using any number of techniques known in the art. In a number of embodiments, such anatomical data may be determined from a two-dimensional or three-dimensional photo, video or model. In a number of embodiments hereof, a characteristic (that is, a dimension such as radius or diameter, or a function thereof) of a patient's or subject's iris maybe used as a dimensional reference in determining anatomical characterization data of a patient's head/face. A non-anatomical reference of known dimension may alternatively be used. However, the use of the iris as a reference point or feature, eliminates the need to use an artificial, external or non-anatomical object having known dimension as a reference in connection with a two-dimensional image and eliminates the need to use an expensive or cumbersome three-dimensional imaging device. The iris provides an anatomical frame of reference on a patient's head/face (certain dimensions of which are important in, for example, determining an appropriate PAP mask) for almost all patients. The diameter of the iris is in the range of 11-13 mm across the human population with no significant differences between the right and left eye, no significant difference in gender, and no significant difference in the ages 5 years and older. Thus, using a diameter of 12 mm for the iris as a reference results in an error of 1 mm (that is, less than 10%), which provides a degree of accuracy at least as good as the use of external or non-anatomical references in a two-dimensional image and, once again, avoids the difficulties associated with acquisition of three-dimensional images. A two-dimensional image or video may, for example, be analyzed via an image characterization algorithm (for example, a computer vision algorithm as known in the art) to determine the iris dimension (for example, radius or diameter) and other physical attributes.

One may collect all of the desired anatomical characterization data using, for example, a photograph from any electronic device with a two-dimensional camera, thereby allowing anyone to take the desired photographs provide such anatomical characterization data without special training or application of a non-anatomical dimensional reference. A two-dimensional image may, for example, be obtained from a patient via an internet-enabled device to collect a complete image of, for example, a patient's face, ears and head. From such data, a series of data metrics (which may evolve over time) are collected regarding the anatomical characteristics of the patient as illustrated in the representative screen capture of FIG. 1.

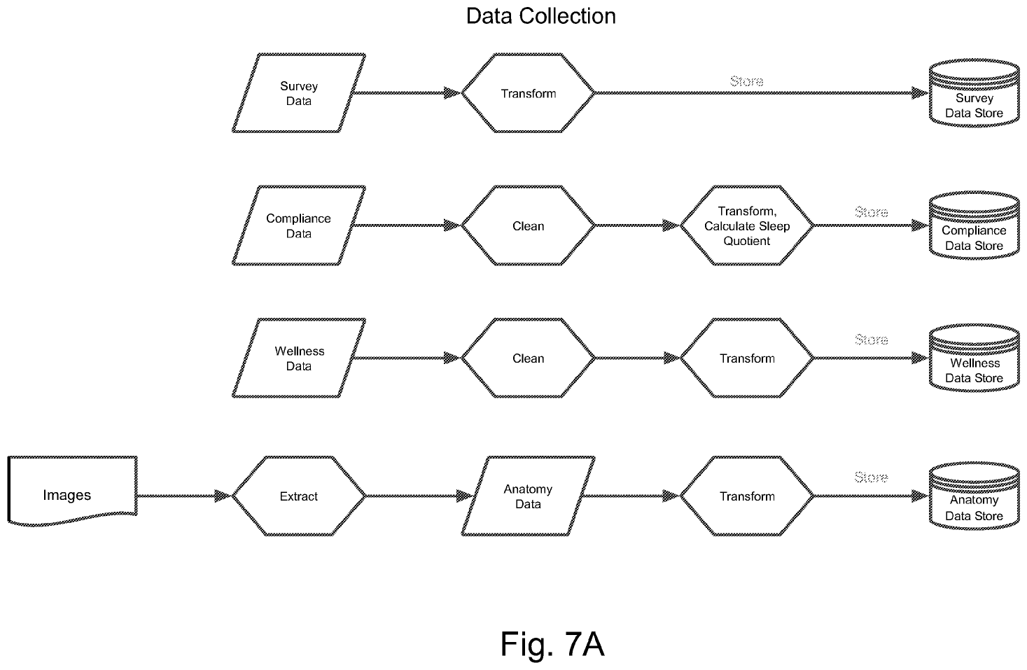

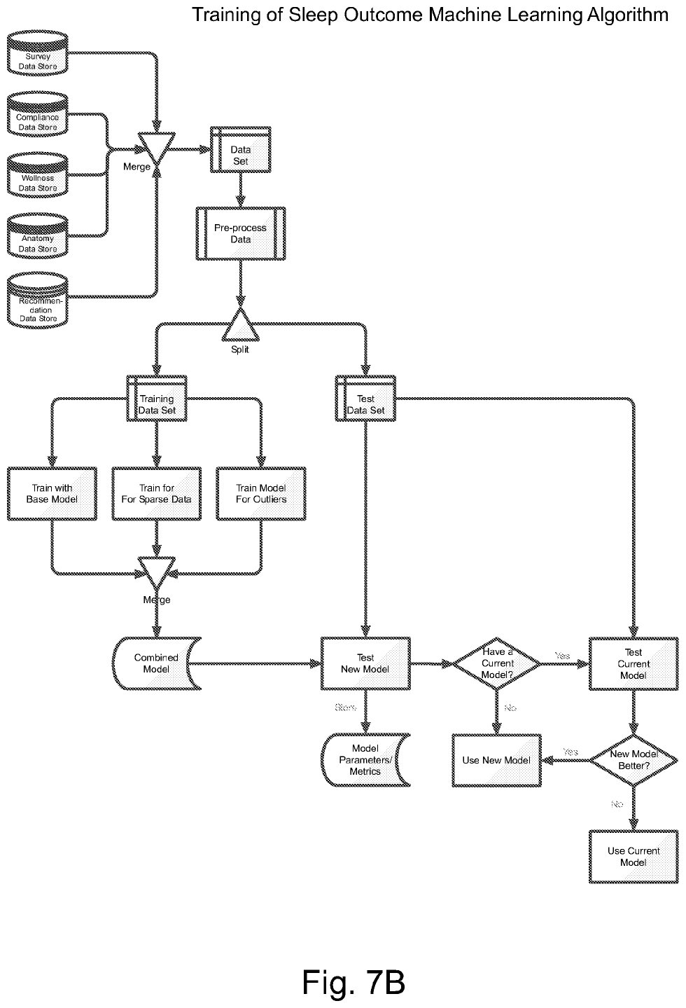

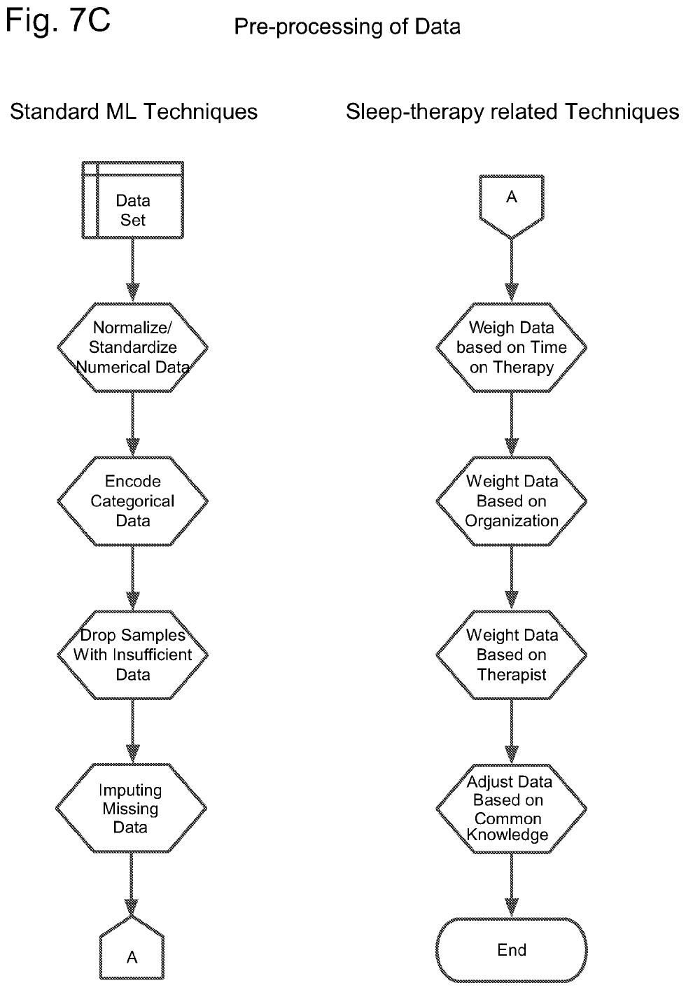

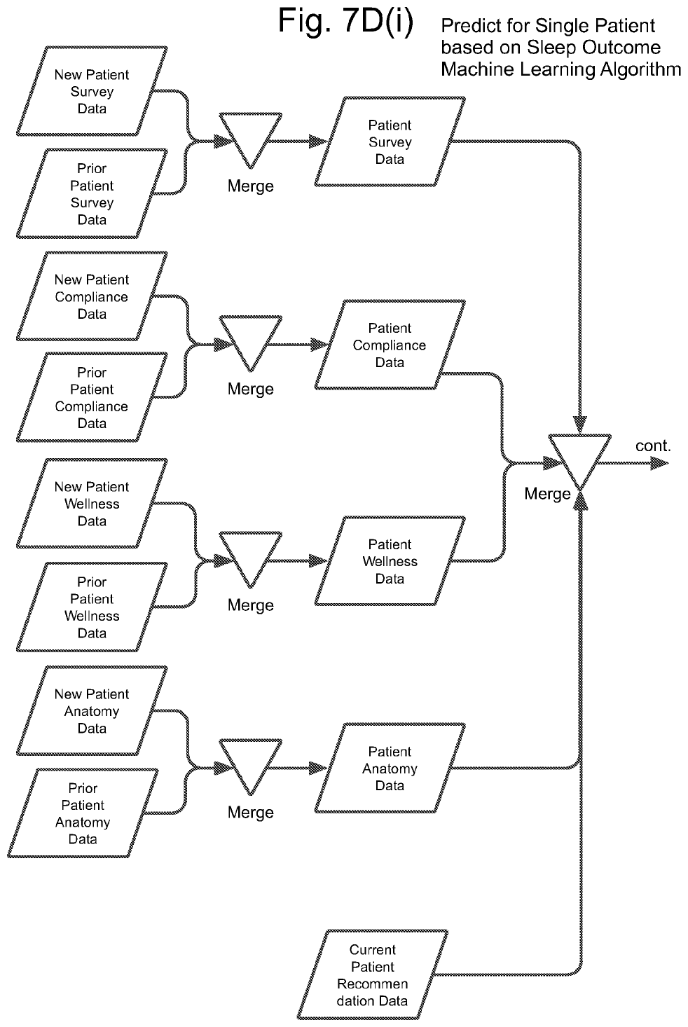

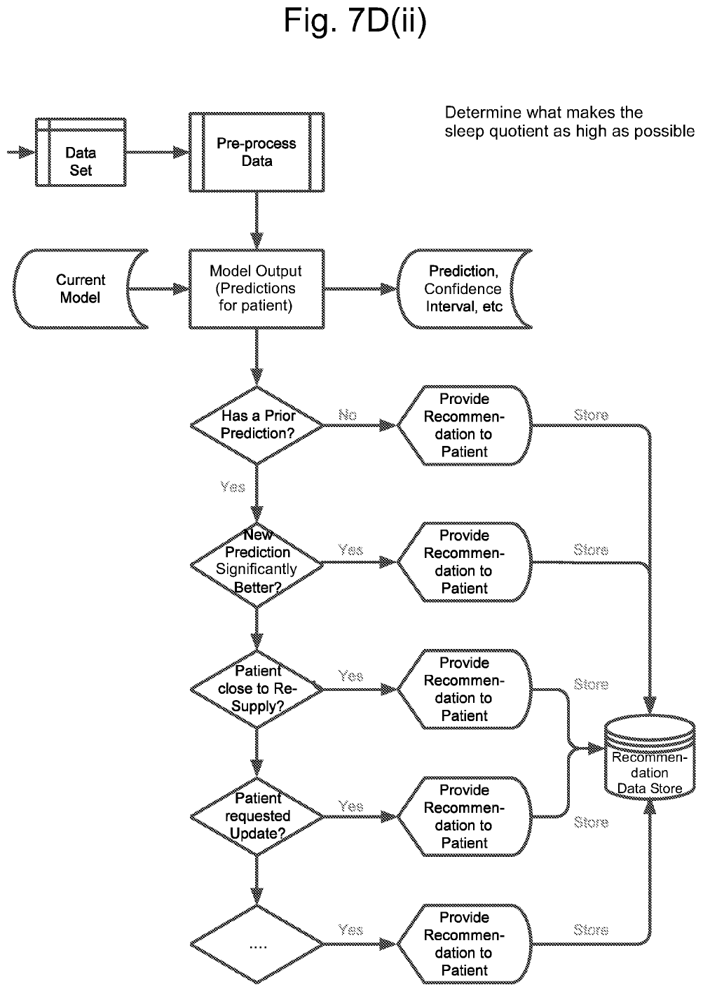

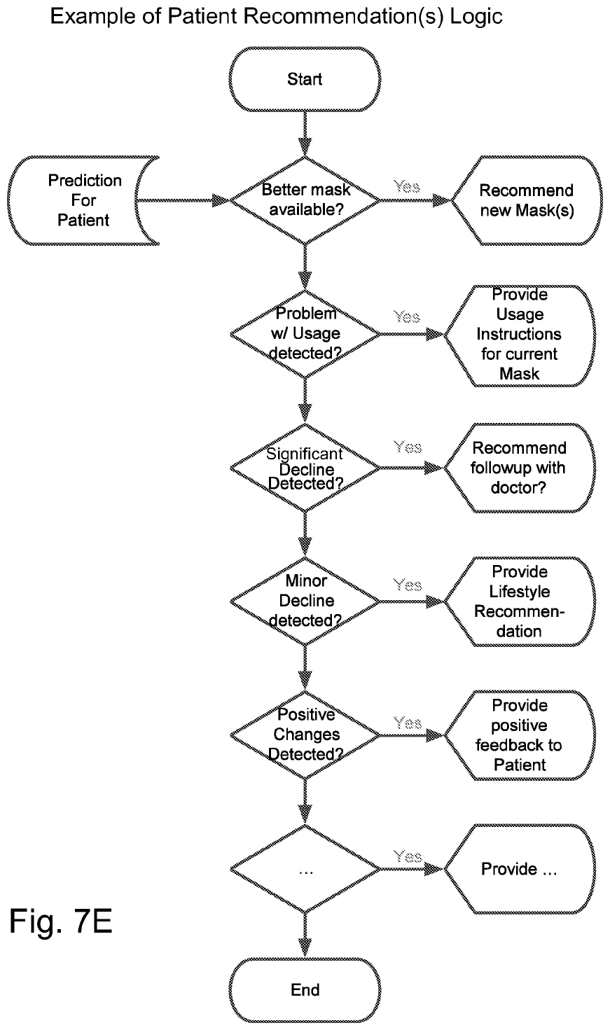

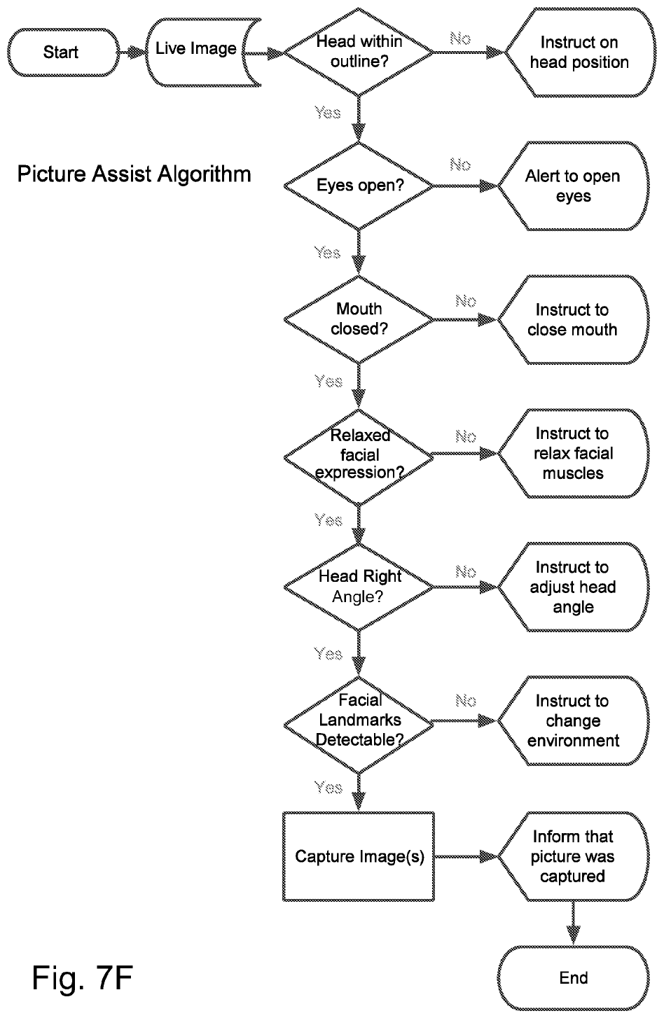

Automated guidance may be provided which evaluates the image in real time and ensures that all pertinent dimensions are collected. Such guidance may include, but is not limited to, aligning the patient inside the outline of a head or other guide, determining that the patient's eyes are open, ensuring that the patient has natural mouth position (for example, not excessively open), ensuring the patient's head is appropriately angled with respect to the camera lens, etc. In a number of embodiments, guidance may, for example, include providing a superimposed outline of a person's head as part of the device and an associated software application via which the photograph is being taken (for example, a personal communication device or computer). Real-time feedback may, for example, be provided as illustrated in FIG. 7F via haptic, audio, or visual feedback/clues to indicate that the patient is or is/is not properly aligned. Additional real-time haptic, audio, or visual feedback/clues may be provided by the system to guide the patient in providing the optimal picture for the extraction of the desired metrics. Such guidance information may include, but is not limited to, ensuring that the patient's eyes are open, ensuring that the system can recognize the patient's iris or other dimensional reference, determining whether the patient's mouth is or is not closed, determining whether facial contrasts are too high because of the surrounding light, etc.

Similar to currently practiced methodologies, a sleep study may first be performed on either a new patient (that is, a person to be newly added to a database of a system hereof, who may or may not be currently using sleep therapy) or on a current sleep treatment patient (that is, a person who is one of the plurality of patients currently monitored by a system hereof). After the sleep study is performed and obstructive sleep apnea has been diagnosed or confirmed, a patient may be accessed, for example, via a communication- (for example, internet-) enabled device which is capable of taking, for example, a standard two-dimensional photographs (or videos) as described above including, but not limited to a tablet, a computer, a smartphone or a dedicated kiosk. The patient may be asked a series of questions to obtain characterization and outcome data, if available. A two-dimensional photograph may also be taken as described above, which is, for example, transmitted/uploaded to a remote processor/analysis center as illustrated in FIG. 2 for analysis (for example, via a software algorithm stored in memory) to characterize anatomical features of the patient using the patient's iris as a dimensional reference as illustrated in FIG. 1.

The anatomical data obtained from a two-dimensional photograph or video using, for example, the iris as a dimensional reference may be used in many methodologies and systems. Such anatomical data may, for example, be used in connection with any system, device or method in which one or more dimensions of an individual's body is an important parameter (for example, in a number of previously practiced methodologies for selecting/fitting PAP masks, for selecting/fitting medical devices other than PAP devices, for selecting/fitting non-medical headgear, for selecting/fitting clothing, for medical diagnostics etc.). If required or desirable, multiple photos may be obtained (for example, from one or more angles or over one or more portions of the body) wherein the iris dimension may be used as a reference to determine other dimensions on the individual's head/body.

In FIG. 2, a patient is illustrated wearing an interface system which is a mask system. In mask system 700, pressurized air is delivered to the patient through a face mask 710 which is connected to the blower of the PAP device via flexible tubing. In mask system 700, face mask 710 (of a size determined for the patient) is retained in connection with the patient's head/face via headgear 720, the fit of which is adjustable. A sealing cushion 712 of mask 710 seals against the patient's face under force applied by headgear 720. In the case of a patient having an interface system (for example, a patient already in PAP therapy), a video or a photograph of the patient wearing their currently supplied interface system (for example, a mask system as illustrated in FIG. 2) may be obtained and the interface system or a portion thereof may be used as a known dimensional reference. In that regard, the interface system data (type, brand and/or size) may be determined using, for example, computer vision software as known in the computer arts. A portion of the interface system of know dimension may then be used as a dimensional reference. Patient anatomical data/metrics may, for example, be determined using the dimensional reference provided by the interface system. Moreover, the data on the interface system (type, brand, size, fitting setting etc.) determined from one or more videos/photos may be used in the characterization data used in the algorithms hereof in, for example, the case of a new patient.

Once again, and as further illustrated in FIGS. 3A and 3B, characterization data associated with each of a plurality of patients is stored in one or more databases of a database system which is stored in a memory system. Patient clinical outcome data from the sleep therapy associated with each of a plurality of patients is also stored in the one or more databases of the database system. The memory system is in operative connection with a processor system (for example, comprising one or more processors). The memory system also includes an algorithm or algorithms executable by the processor system to process and/or analyze the data of the database system to manage the treatment of sleep therapy patients. The algorithm(s) may, for example, be executable to select (or to recommend) a therapy management option (for example, an oral/dental device, a PAP device, a type of PAP device), to select an interface system for a patient (for example, an initial interface system for a new sleep therapy patient or a change in interface system for an existing sleep therapy patient) from a plurality of different interface systems, to fit a selected interface system to a patient (for example, to determine a size of a mask system and/or to determine settings for associated headgear), to monitor a patient after therapy selection and interface/mask system selection and fitting, and to provide overall management and/or improvement/optimization of the patient's continuing sleep treatment. As discussed further below, the algorithm(s) hereof may include one or more machine learning algorithms or subalgorithms, methods or procedures. As known in the computer arts, components of software hereof may reside on a single computer or be shared/distributed among multiple computers.

As described above, one or more outcome parameters are defined via one or more outcome metrics (that is, clinical outcome data metrics indicative of the efficacy of the PAP therapy) associated with therapy management options (for example, use of particular therapy, interface system etc.) for each of the plurality of patients to represent a quantification of a patient's outcome/results in sleep therapy. Such outcome parameters and the variables metrics associated therewith may be analyzed for improvement or optimization of a new or existing patient's sleep treatment in the algorithm(s) hereof through selection/changes in management/treatment options or variables (for example, interface selection (for example, type, brand, and size), headgear options, mask headgear fitting, humidification, type of therapy, pressure prescription, etc.).

With respect to the representative example of selection of an interface system from a plurality of different interface systems, unlike other attempts at automating a PAP interface/mask system selection and fitting process, the devices, systems and methods hereof do not attempt to determine or optimize interface system selection/fitting on the basis of, for example, matching interface/mask physical characteristics/dimensions with anatomical features of a patient's head/face. To the contrary, in a number of embodiments hereof, one or more quantified patient outcome parameters are predictively optimized for a given patient via the algorithm(s) hereof on the basis of the data metrics available for the given patient. Interface selection and interface fitting are among the variable/metrics used in determination or predicted of an optimization of one or more outcome parameters. In a number of embodiments, models from one or more machine learning algorithms, previously trained using patient characterization data metrics and patient outcome data metrics (which are determined, from previous sleep therapy treatment for the plurality or population of patients) in the database system hereof are used in determining/optimizing one or more predicted outcome parameters.

In the case of selecting an interface system for a new sleep therapy patient or a selecting a change in an interface system for an existing sleep therapy patient, one or more such outcome parameters (or functions thereof) may be predictively optimized for the patient by predictively determining or selecting the interface (for example, a mask) most likely to result in optimization of the outcome parameter(s). In general, outcome parameters for a given patient may be predictively optimized for a given patient in, for example, selecting an interface system based upon the data available for the patient at the time of determination, which may be limited. In the case of, for example, selecting an interface system for a new patient, patient characterization data will be available (from anatomical characterization and patient/therapist queries or surveys) but no clinical outcome data may be available. If clinical outcome data are available for a new sleep therapy patient (for example, from a sleep study or previous treatment), such data may be used in determining input metrics. Management options other than interface selection are determined or selected in a similar manner.

As set forth above, algorithm(s) hereof may include one or more machine learning algorithms or subalgorithms which have been trained on patient characterization data and clinical outcome data for a plurality of sleep therapy patients previously entered in the database(s) hereof. The data available for the new patient is entered into the database and the model or models resulting from machine learning algorithm(s) identify, select or predict an interface system (and/or other sleep therapy management option(s)) determined on the basis of the interface system (and/or other sleep therapy management option(s)) being mostly likely to provide a maximized or optimal result for the one or more predefined outcome parameters. In a number of embodiments hereof, an interface system such as a mask system is selected solely on the basis of such an optimization determination. Once again, although anatomical data is a component of the patient characterization data metrics used in the algorithms hereof, an interface (for example, mask) system is not selected on the basis of matching physical characteristics of the interface system with anatomical features of a patient's head/face in a number of embodiments hereof.

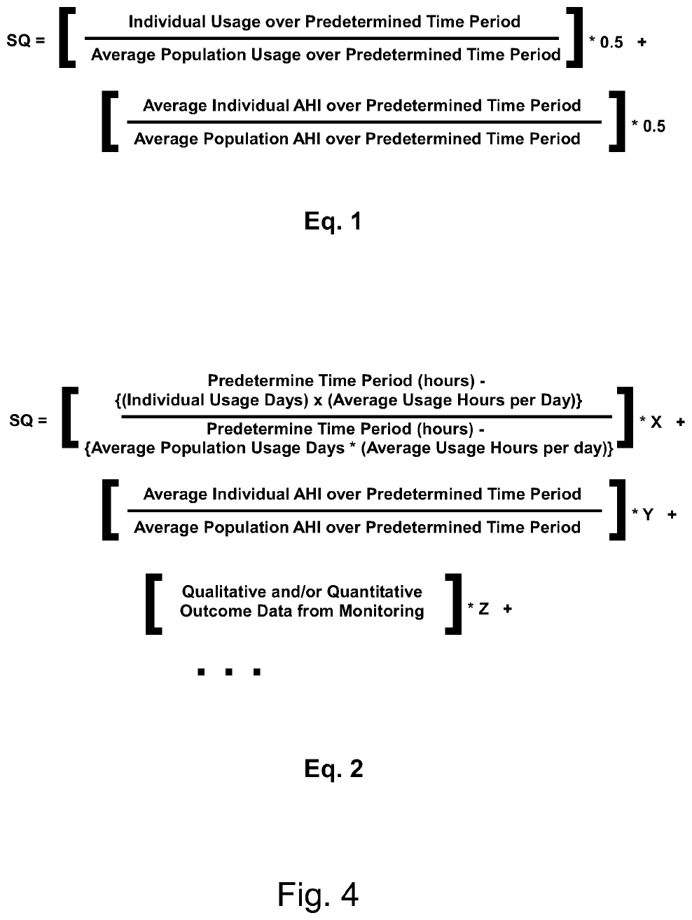

Equations 1 and 2 (Eq. 1 and Eq. 2, respectively) of FIG. 4 set forth representative embodiments of a patient outcome parameter (sometimes referred to herein as a Sleep Quotient™ or SQ™) for each of the plurality of (sleep therapy) patients in a database hereof which may be determined based on data of clinical/behavioral outcome metrics. In the embodiment of Equation 1, the outcome parameter SQ is a function of two outcome metrics: the measured apnea hypopnea index or AHI (a measure of the average number of apnea events in one hour during sleep) and the actual cumulative time of usage by the patient of the treatment over a predetermined period of time. Each of the AHI and time of usage components of the equation are weighted equally in Equation 1. In Equation 1, the usage component is determined by dividing the usage time for the individual patient over a predetermined period of time by the average population usage time over the predetermined period of time (that is, the average usage time for all the patients in the database system over the predetermined period of time). The AHI component is determined by dividing the average AHI for the individual patient over the predetermined period of time by the average AHI for the population over the period of time. Each of the usage component and the AHI component are multiplied by the same weighting component of 0.5 in the representative embodiment of Equation 1.

In general, if a treatment protocol is successful in reducing the AHI to less than a predefined number of apneas per time (for example, to less than five apneas per hour, on average) and the patient uses the therapy for a predetermine period of time per night (for example, seven to eight hours per night), it is likely that the patient will have enjoyed a healthy night of sleep. Achieving a significant reduction in AHI is desirable but not sufficient in and of itself to achieve an acceptable/optimal outcome. It is also necessary to achieve adequate time of usage by the patient over a predetermined period of time.

In the embodiment of Equation 2, additional qualitative and/or quantitative monitoring data metrics are included in the determination or defining of SQ. Weighting factors X, Y, Z etc. may be used to adjust the effect of each component of the SQ on the overall value thereof. Such weighting factors and/or other characteristics for outcome parameters hereof such as SQ may be changed via improvement/optimization of the algorithm over time with the addition of more patients and/or data into the database system. In the usage component of Equation 2, the product of individual's usage days and the individuals average usage hours per day over the predetermined period of time is subtracted from the total predetermined time period in units of hours (for example, 30 days*24 hours/day or 720 hours for the case that the predetermined period of time is 30 days) to determine a numerator. The denominator is determined by subtracting a product of the average usage day for the population and the average usage hours per day for the population from the total predetermined period of time. As clear to those skilled in the art, the predetermined period of time need not be 30 days.

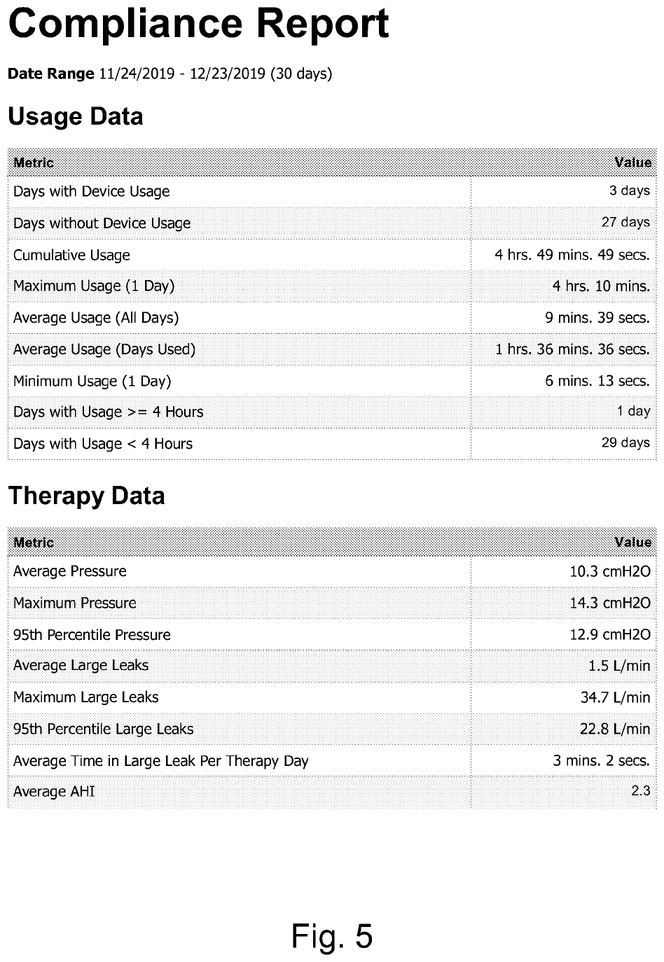

In a number of representative embodiments, data metrics used to define or calculate the parameter SQ (as well as other potential outcome parameters for user herein) is taken from three sources. First, data is taken or determined from metrics output by the PAP (blower) device which, for example, records apnea events as well as usage time. Average or maximum mask leak is also measured by the PAP device and may be used in determining SQ (and/or other metrics) in embodiments hereof. FIG. 5 illustrates a representative example of a compliance report generated from data of a PAP device which includes data/metrics that may be used in determining SQ and/or another patient outcome parameter. Secondly, qualitative metrics may be derived from patient-provided information which may, for example, come from self-reported information or from responses to inquiries to the patient. For example, context or situations sensitive questions (for example, a level of drowsiness while driving as determined by a questionnaire administered shortly after cessation of driving—for example, as characterized over a range of 0 to 10; or if the patient had any trouble with sleep therapy during last night's sleep) as discussed further below may be used in determining SQ. Other outcome metrics may also or alternatively be used including, but not limited to, metrics measuring the frequency of masks to be exchanged as a result of patient compliance issues, patient discontinuance of treatment as a result poor compliance, lack of satisfaction, disappointing clinical results etc.

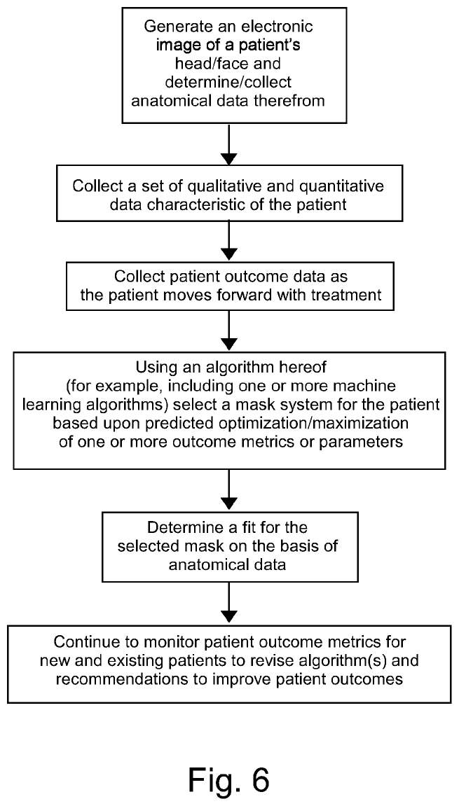

FIG. 6 illustrates a logic flow chart of a representative example of a course of actions in a methodology hereof for a patient being added to a sleep therapy management system hereof for whom sleep therapy is currently being provided. As described above, the stored/executable algorithm(s) hereof used to determine outcome-based therapeutic management options (for example, interface/mask system selection) may, for example, include one or more artificial intelligence (AI) and/or machine learning algorithms, subalgorithms, procedures or methods. The machine learning algorithm(s) may be trained (for example, on the basis of known data of expert therapist/clinician decisions in treatment/management options such as mask system selection) using collected patient characterization and patient outcome data, including, but not limited to data from anatomical measurements, data from questionnaires/surveys, data from PAP devices, and data from other sources as described above.

A recommendation/determination system of the algorithm(s) hereof may, for example, use multiple machine learning algorithms to obtain better predictive performance than could be achieved using any one of the machine learning algorithms by itself. Representative examples of individual machine learning algorithms suitable for use herein include, but are not limited to, Linear Classifiers (Logistic Regression, Naive Bayes Classifier, etc.), Support Vector Machines, Kernel Estimators (k-Nearest Neighbor, etc.), Decision Trees (Random Forests, etc.), Boosting Algorithms (Ada Boost, Gradient Boosting, XGBoost, etc.), and Neural Networks. In one embodiment XGBoost is used as the base machine learning algorithm, Random Forest is used as the machine learning algorithm for sparse data, and the Naive Bayes Machine Learning algorithm is used for outliers. Each of the individual machine learning algorithms is trained by the full or a subset (based on the criteria applicable to the selected machine learning algorithm) of training data set. In a number of embodiments, the individually employed machine learning algorithms and models are combined into one single machine learning algorithm and model. A machine learning model is the output generated when “machine learning algorithm” is trained with the training data set. In a number of embodiments the combined algorithm/model outputs predictions for each of the individual algorithms and selects the model prediction with the best outcome best confidence interval (for example, the interval with the highest value or the mean of the confidence finite interval endpoints). In a number of other embodiments, the combined algorithm/model outputs predictions by combining weighted results of the individual algorithms. In still other embodiments, the combined algorithm/model may use only a subset of the individual algorithms/models based on properties of the data (for example, the combined machine learning algorithm selects the machine learning algorithm and model for sparse data when the input data has a large amount of missing attributes). The machine learning algorithm/model is evaluated against a prior determined test data set on a set of evaluation metrics including, but not limited to, accuracy, precision, learning performance, and prediction performance. This process is illustrated in FIG. 7B. FIG. 7B also sets forth an embodiment of a methodology for testing new models (that is, an updated model using the current machine language algorithms or a model output from a different machine learning algorithm or combination thereof than present in the current algorithm(s)/model). In the case of a system already including a machine learning algorithm/model (once again, the output of which is sometimes referred to as the current model), the existing model is evaluated against the same test data set as the new model. Both machine learning models (or algorithm/model combinations) are evaluated against each other based on a weighted formula of the collected metrics and the better performing of the two models is used for the system as illustrated in FIG. 7B.

In a number of embodiments, the algorithm(s) hereof may, for example, be configured to ensure that more recent data is prioritized over older data. The algorithm(s) hereof may be optimized for prediction accuracy and speed to deliver recommendations with the highest probability of the patient adhering to the sleep therapy in near real-time. The algorithm(s) hereof are constantly retrained and reevaluated based on newly collected patient data to provide updated models.

In a representative embodiment, a boosting algorithm such as XGBoost is used as a basis for the machine learning algorithms hereof. In general, a boosting algorithm is an ensemble meta-algorithm which can reduce bias and variance in supervised learning. Boosting machine learning algorithms increase the strength of learning algorithms. Boosting algorithms may, for example, be used to convert a set of weak learning algorithms into a single, stronger learning algorithm. Strengthening a learning algorithm increases the correlation of a classifier with the true classification. XGBoost is a machine learning algorithm which is operated under a gradient boosting framework and provides a parallel tree boosting. Gradient boosting is a machine learning technique used in regression and classification problems. It produces a prediction model in the form of an ensemble of weak prediction models, which are usually decision trees, and builds the model in a stage-wise fashion. Gradient boosting generalizes models by allowing optimization of an arbitrary differentiable loss function. XGBoost uses the Apache License, which is a permissive, free software license prepared by the Apache Software Foundation. The Apache License permits users to use the software for any purpose, including, distributing, modifying, and distributing modified versions of the software under the terms of the license, without concern for royalties. The core XGBoost algorithm was not modified in use in studied algorithms hereof.

A decision tree algorithm such as a random forest or random decision forest algorithm may be used to account for noisy data and to deal with any observations including a large amount of missing values. Random decision forests algorithms provide an ensemble machine learning method for classification, regression and other operations which construct a plurality of decision trees during training and output a class that is the mode of the classes (classification) or the mean prediction (regression) of the individual trees.

Further, in the above, representative embodiment, a classifier such as a naive Bayes classifier may be used to better handle patients that are outliers when compared to most of the others of the plurality of patients/population. In general, naive Bayes classifiers provide a simple technique for constructing classifiers (that is, models that assign class labels to problem instances represented by vectors of feature values, wherein the class labels are drawn from some finite set). There are a family of algorithms for training such classifiers based on a common principle that the value of a particular feature is independent of the value of any other feature, given the class variable.

Before the data is used to train a recommendation/management algorithm hereof, it may be cleaned, pruned, and transformed (as known in the computer arts) based on criteria designed for the sleep therapy problem domain. As illustrated in FIG. 7C, the cleaning, pruning, and transforming may, for example, include standard machine learning data pre-processing techniques, such as, but not limited to, normalization/standardization of numerical data, encoding of categorical data, and dropping or imputing missing data. Additionally, techniques related to the sleep-therapy domain may be used to further clean, prune, and transform the data. Such techniques include, but are not limited to, weighting of data based on the time the data was recorded relative to the start of the patient's therapy or based on the person reporting/collecting the information. In a representative embodiment, the early compliance data for patient (for example, in the first week of the sleep therapy) is weighted lower, since the patient is still adjusting to the new therapy and the data is less indicative for the long-term success of the sleep therapy. In another representative embodiment, the survey data reported by a sleep therapist is weighted higher than the data reported the patient to account for the therapist knowledge in sleep therapy sciences. In another representative embodiment, the data reported by all therapist for a particular organization is weighted lower to account for the bias introduced by the therapists training in the particular organization.

As illustrated in FIGS. 7D(i) and 7D(ii), the current machine learning algorithm/model is used to predict values for variables, parameters or metrics characterizing a patient's sleep therapy which predictively optimize one or more outcome parameters for a single patient based on the individual patient's newly collected data (new patient data in FIG. 7D(i)) and any historical data for the patient (prior patient data in FIG. 7D(i)), if available. Such data is collected from various sources (for example, questionnaire/survey data, data from uploaded patient photographs, data from a PAP device) and includes patient characterization data (for example, anatomical data, health wellness data, behavioral data etc.) and data on measured outcome metrics (for example, compliance data, level of drowsiness during and/or or after specific activities, etc.). The data from the different databases or stores are merged into one dataset, cleaned, pruned, and transformed in the same or similar fashion and format as the data used to train the machine learning algorithm.

In a number of embodiments, together with providing sleep therapy recommendations (for example, a recommendation of a type of therapy such as PAP, CPAP, BiPap and a mask system selection for new patients as well as continuing recommendations for changes/improvement during ongoing therapy) based on model predictions, the algorithm(s) hereof may determine or calculate other parameters such as, but not limited to, a confidence interval of the patient's adherence to the recommended sleep therapy on the basis of analysis of the patient characterization data and one or more clinical outcome metrics or parameter(s) described herein. The new prediction, together with the associated parameters, is stored in a database system and may be compared to prior predictions and the associated parameters therefor, if available, to provide one or more recommendations to the patient. If no previous prediction exists for the patient, all sleep therapies above a certain confidence threshold may, for example, be considered as options for recommendation to the patient. In another embodiment, a new interface/mask is recommended to the patient if the new prediction (associated with a change in a metric or parameter characterizing the patient's sleep therapy) is above a certain threshold when compared to the current prediction. In that regard, the predicted optimized outcome parameter (for example, SQ) associated the new prediction may be compared to the predicted optimized outcome parameter associated with the current prediction to determine if a threshold has been exceeded. Different thresholds may be established to, for example, characterize the significance of a change in prediction which may, for example, be used to determine the timing of a recommendation for change provided to the patient. A new model prediction for an individual patient may not only triggered based on newly collected data for the patient, but may also be triggered based on one or more other factors such as, but not limited to, an update to the current machine learning model, the availability of the new interface system from a manufacturer or a patient request. New model predictions may also occur continuously, but this would require significant computational resources.

Care is taken in pre-pruning of the data of the database system hereof based on knowledge in sleep therapy problem domain. Individual therapists may, for example, add bias to their selection process based on training, company policy and procedures, as well as their own experience. Moreover, bias may arise in the way patients answer sleep, wellness, and other questions based on their experience, or lack of experience, with sleep therapies, etc. Examples of adapting the algorithm to the sleep domain extend from the simple, such as limiting the solution space by not considering masks with magnets for patients with a pacemaker, to the more complex, such as independently training the algorithm for different organizations and or segments of the population. For example, independent training may occur on the basis of patients living in certain regions and/or patients being in a certain age group/generation. In a number of embodiments, an individual algorithm may be executed for a specific patient matching the criteria therefor or a combination of individually trained algorithms may be executed for a specific patient.

Referring, for example, to the representative logic flow chart of FIG. 6, one or more two-dimensional photographs may, for example, uploaded to the system hereof as described above. Anatomical patient characterization data may, for example, be determined from the photograph(s) using the iris as a dimensional reference as described above. As also described above, other quantitative and qualitative patient characterization data is collected and input into the database system hereof. Available patient outcome data in connection with currently used sleep therapy equipment may also be uploaded. As illustrated in FIG. 7A, the data is cleansed, transformed, etc., and derived metrics such as, but not limited to, the Sleep Quotient are calculated. In one embodiment, incoming compliance data is converted from hours, minutes, and seconds to seconds. In another embodiment, missing fields are filled with default values, such as unknown. One or more machine learning algorithms as described above may, for example, be used to select a new mask system (based upon predicted maximization/optimization of one or more outcome parameters as described above), which may also be fitted for the patient via a machine learning model prediction or using anatomical data. As compared to current practices or methods for mask system selection and fitting, improved results are achieved via linking the determination or management options to clinical outcome metrics based upon analysis of data from a plurality of patients stored in the database system hereof. Once again, the database data is continuously supplemented with new data and the associated algorithm(s)/models are continuously trained/improved through addition of data of new patients and/or the input of additional data from monitoring of existing patients. In the case of a mask system, the mask system may, for example, be delivered to the patient using standard means. The patient's headgear may be preadjusted to a predetermined fit (from patient anatomical data) so that no adjustments are required when the mask system arrives, thereby eliminating the need for an in-person fitting.

When the mask is received by the patient, a targeted video may be transmitted to the patient, for example, via a software application hereof which may be stored and executed upon a personal communication device of the patient to provide instructions on donning the mask system, using the mask system in sleep therapy, cleaning the mask system etc. Moving forward, the patient outcome data/metrics for that patient are monitored in connection with use of the selected mask system. The collected data on patient outcome metrics are transmitted to and included in the database for use in managing and improving sleep therapy (including making adjustments or changes in sleep therapy options/variable such as mask system, initiating or changing humidification etc.). The collected clinical outcome data are also used in training/developing the algorithms hereof for all patients (including, in making recommendations for changing management options such as selecting a new mask system for such patients). Intelligent and targeted troubleshooting is enabled when necessary based upon monitoring data and clinical results. Information in the form of text, audio and/or video, which is highly targeted to a particular patient and the associated mask system may be transmitted to the patient in a timely manner.

In addition to initial selection, fitting and continuous improvement/optimization of the mask system, the systems hereof are thus functional to troubleshoot existing interface systems by interpreting how the interface system is being used. This ongoing capability is an important component in achieving favorable outcomes and long-term compliance. As illustrated in FIG. 7E, in the (continuous) monitoring algorithm hereof, a calculation is determined for one or more patient outcome metrics such as SQ and a determination is made of what information (for example, a recommendation or recommendations) is communicated to the patient (if any) at a particular time. In one embodiment, the current machine learning model predicts that a better mask system is available based on newly acquired patient data, an updated prediction of the latest machine learning model, and/or any newly released mask systems. If the magnitude of the change in the predicted effectiveness (for example, based on the change in predicted outcome parameter such as SQ) when compared to historical data on such metric(s) is greater that one or more predetermined thresholds, a recommendation for action may be made. In a representative embodiment, if the predicted change in the outcome parameter is greater than a first predetermined (higher or more severe) threshold, a recommendation is made to change the mask system as soon as possible. If the change is greater than a second predetermined (lower or significance) threshold, but not greater than the first threshold, it may be recommended to wait (for example, until the next re-supply interval) to change the mask system.

In another embodiment, the current machine learning model determines or predicts continued use of the currently used interface system for a patient, but the latest model calculation, based on a the time-series of historic and currently measured metrics, predicts a future decline in the one or more outcome parameters patient's (for example, SQ and/or one or more other outcome parameters) for a patient below a predetermined first (higher) threshold value but not below a predetermined second or separate (lower) threshold. In this situation, the system may, for example, provide usage instruction or reconfiguration options (such as adjusting the sizing of the headgear, changing the headgear, changing or replacing a mask seal, etc.) to the patient. As clear to one skilled in the art, different actions may be determined based on more than two thresholds in the case of a decline in one or more outcome metric(s).

In another embodiment, the current machine learning model continues to predict use of the currently used mask for the patient and the calculated SQ for the patient has been found to be constant or increased over a period of time. In such a circumstance, the system may, for example, provide the patient with positive feedback/reinforcement and encouragement to continue that the current sleep therapy.

In the troubleshooting process, a patient may, for example, initiate a request for help or be proactively prompted via a mobile application hereof or via a website associated with the systems and methods hereof. In communicating with a patient, voice assistants like GOOGLE® VOICE® (available from Google LLC of Mountain View, Calif., United States), AMAZON® ALEXA® (available from Amazon.com, Inc. of Seattle, Wash., United States) and APPLE® SIRI® (available from Apple, Inc.) may be used to interact with the patient.

A series of questions may be asked to identify any issue being experienced by the patient. Periodically, based upon data from the PAP device, monitoring data from a personal communication device (for example, a smartphone) or one or more wearable devices (for example, APPLE Watch, FITBIT, etc.), including, for example, behavioral data determined by sensing that the patient has awoken after sleeping or has just finished a task such as driving, the system hereof may collect quantitative and qualitative data indicative of the quality of the patient's sleep therapy. The patient may, for example, be asked to take a photo or series of photos (for example, using personal communication device or any electrical device with a camera) while wearing their PAP interface system as described above. Such a photo or photos may, for example, provide the following information: (a) the mask system itself provides a known reference point (dimensional scale) which allows the software to extract quantitative dimensional information about the patient and the patient's use of the mask; (b) by uploading a photo of the mask system while being worn by the patient and utilizing computer vision and machine learning together, the algorithm(s) of the system hereof can, in many instances, identify ways that the patient may be using the device incorrectly or non-optimally and recommend changes or adjustments to the current mask; and (c) the algorithm(s) of the system hereof may recommend a new or different mask as appropriate.

As described above, in a number of embodiments hereof, one or more software applications or apps may be provided for access by a patient which is executable on, for example, a personal communication device or other computer to communicate information between the patient and a remote system. Such an app can, for example, be used to facilitate initial communication of patient characterizing data to the remote system as well as follow-up communication with a patient once a mask system is selected and treatment begins. In a number of embodiments, a context or situation sensitive questionnaire may be communicated to the patient via the app to prompt the patient to transmit (for example, via a personal communication device or other communication enable computer) information relevant to outcome metrics associated with the patient's use of a selected mask system. In that regard, the timing of and/or the nature of the questions of the questionnaire can be determined based upon recent actions or experiences of the patient. Such situation sensitivity may, for example, be determined from data (for example, sensor/use data) from the personal communication device and/or other device used (for example, worn) by the patient. In a representative example, data from a personal communication device and/or at least one other device used by the patient may be used to determine that the patient is likely to have recently participated in a predetermined activity (for example, sleeping, driving etc.), and the questionnaire may include at least one question inquiring of a level of drowsiness of the patient during or immediately after the activity. Context or situation sensitivity may, for example, be determined from data including one or more of motion data, time data, use/nonuse data and/or location data.

The high-level nature and complexity associated with the determinations, methods, and systems described herein, including, for example, the multiple and varied combinations of patient data, therapy options, computations, calculations and determinations cannot be done in real time, quickly or at all by a human. The processes described herein thus are dependent upon the machines described herein.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A method of managing sleep therapy, comprising:
inputting characterization data associated with each of a plurality of patients into a database system stored in a memory system;
inputting outcome data associated with each of the plurality of patients resulting from use of one of a plurality of interface systems worn during sleep therapy;
training at least one machine learning procedure of an algorithm stored in the memory system and executable via a processor system using a training set of the characterization data associated with each of a plurality of patients and the outcome data associated with each of the plurality of patients resulting from use of one of the plurality of interface systems to create at least one machine learning model,
determining a management option for sleep therapy for a person via execution of the algorithm based at least in part upon an optimization of at least one outcome parameter for the person predicted by the at least one machine learning model based at least in part upon characterization data of the person input into the database system, wherein the management option comprises at least one of selection of an interface system from the plurality of interface systems for future use, fitting of a selected interface system for future use, and a change in use of a currently used interface system; and
communicating information regarding the management option to the person.

2. The method of claim 1 wherein the person is one of the plurality of patients or is a new patient additional to the plurality of patients.

3. The method of claim 2 wherein characterization data of the person and any available output data is input into the database system and the management option for sleep therapy determined via the predicted optimization of the at least one outcome parameter based at least in part upon characterization data and the outcome data of the person input into the database system.

4. The method of claim 2 wherein the algorithm comprises a plurality of machine learning procedures or a plurality of machine learning models.

5. The method of claim 2 wherein the at least one outcome parameter is a function of outcome metrics comprising a number of apneas over a predetermined period of time and a usage time over the predetermined period of time.

6. The method of claim 5 wherein the outcome metrics further comprise a level of drowsiness after a defined activity.

7. The method of claim 2 wherein at least a portion of the outcome data is determined from at least one of data measured from a PAP device, questionnaire data or observed patient behavior.

8. The method of claim 2 wherein each of the plurality of patients is monitored over time and new data comprising at least one of new characterization data or new outcome data for each of the plurality of patients is input into the database system over time.

9. The method of claim 8 further comprising updating training of the algorithm based upon the new data for each of the plurality of patients to create at least one updated machine learning model.

10. The method of claim 9 further comprising testing each of the at least one updated machine learning model and the at least one machine learning model against a test data set to determine which of the at least one updated machine learning model and the at least one machine learning model has a better confidence interval; and using the one of the at least one updated machine learning model and the at least one machine learning model with the better confidence interval to determine the management option.

11. The method of claim 2, wherein the management option comprises a change in a sleep therapy option, and the method further comprises determining if a recommendation to make the change in the sleep therapy option is to be communicated to the patient based upon a predetermined threshold in a change in the predicted optimization of the at least one output parameter.

12. The method of claim 2 wherein the management option further includes at least one of a recommendation for an appointment with a physician, a recommendation for a change in lifestyle, a recommendation for a change in sleep behavior, providing education on sleep therapy, or providing positive feedback.

13. The method of claim 1 wherein the management option comprises a selection of an interface system from the plurality of interface systems for future use.

14. The method of claim 1 further comprising, upon occurrence of a triggering event, determining an updated management option for sleep therapy for at least one of the plurality of patients via execution of the algorithm based at least in part upon an optimization of the at least one outcome parameter for the at least one of the plurality of patients predicted by at least one machine learning model determined for use at the time of the triggering event and based at least in part upon the characterization data associated with the at least one of the plurality of patients and the outcome data associated with the at least one of the plurality of patients.

15. The method of claim 14 wherein the triggering event comprises a passage of a predefined period of time, a request for the at least one of the plurality of patients, receipt of new characterization data associated with the at least one of the plurality of patients or new outcome data associated with the at least one of the plurality of patients, initiation of use of at least one updated machine learning model, use of a new interface system by at least a portion of the plurality of patients.

16. The method of claim 1 further comprising providing a software application on a device of the person which is executable on the device of the person to communicate information between the patient and a remote system including the database system and the algorithm.

17. The method of claim 16 wherein the device is a mobile personal communication device.

18. The method of claim 16 further comprising communicating a questionnaire to the person via the software application that is situation sensitive and inputting outcome data determined from a response of the person in the database system.

19. The method of claim 18 where situation sensitivity is determined from data from the device or at least one other device of the person used by the person and the timing of communicating the questionnaire to the person is based upon data from the device or the at least one other device.

20. The method of claim 19 wherein data from the device of the person or the at least one other device used by the person is used to determine that the person is likely to have recently participated in a predetermined activity and the questionnaire includes at least one question inquiring of a level of drowsiness of the person.

21. The method of claim 20 where situation sensitivity is determined from data comprising one or more of motion data, time data and location data.

22. The method of claim 21 wherein the predetermined activity is driving.

23. The method of claim 2 wherein the characterization data of each of the plurality of patients comprises one or more of anatomical data, sleep behavior data, demographic data, health data, or sleep therapy data.

24. The method of claim 1 further comprising obtaining a video or an image of the person wearing a currently used interface system and the algorithm is further configured to determine if the patient is using the interface system incorrectly or non-optimally and to recommend changes or adjustments in use of the interface system or to change the interface system.

25. (canceled)

26. The method of claim 24 wherein the algorithm comprises a computer vision procedure to assist in at least one of identifying the interface system or in determining if the patient is using the interface system incorrectly or non-optimally.

27. The method of claim 24 further comprising using at least a portion of the interface system is as a reference of known dimension.

28. The method of claim 23 wherein the anatomical data comprises at least one anatomical characteristic of the person's head and the method further comprises:
determining the at least one anatomical characteristic based at least in part on the at least one image or video and a known dimensional reference in the at least one image of video selected from an iris of the patient or at least a portion of a sleep therapy interface worn by the patient in the at least one image or video.

29. (canceled)

30. (canceled)

31. The method of claim 2 wherein the at least one image is a two-dimensional image and the two-dimensional image is analyzed via an image characterization.

32. The method of claim 1 wherein the management option comprises at least one of selection of an interface system from the plurality of interface systems and fitting of a selected interface system.

33. The method of claim 32 wherein the management option comprises determining a fitting for headgear of the interface system so that the fitting of the headgear of the interface system is adjusted to the determined fit before delivery.

34. A system of managing sleep therapy, comprising:
a memory system;
a processor system in operative connection with the memory system;
a database system stored in the memory system, the database system comprising characterization data associated with each of a plurality of patients, and outcome data associated with each of the plurality of patients resulting from use of one of a plurality of interface systems worn during sleep therapy; and
an algorithm stored in the memory system and executable via a processor system, the algorithm comprising at least one machine learning procedure trained using a training set of the characterization data associated with each of a plurality of patients and the outcome data associated with each of the plurality of patients to create at least one machine learning model, wherein the algorithm determines a management option for sleep therapy for a person based at least in part upon an optimization of at least one outcome parameter for the person predicted by the at least one machine learning model based at least in part upon characterization data of the person input into the database system, wherein the management option comprises at least one of selection of an interface system from the plurality of interface systems for future use, fitting of a selected interface system for future use, and a change in use of a currently used interface system.

35.-57. (canceled)
